(19) United States
(12) Patent Application Publication
(10) Pub. No.: US 2013/0167839 A1
(43) Pub. Date: Jul. 4, 2013

(54) COMBINATION LARYNGOTRACHEAL CANNULA AND ENDOTRACHEAL STYLET FOR USE IN GUIDING AN OUTER ENDOTRACHEAL TUBE DURING AN INTUBATION PROCEDURE WHILE PRE-ANESTHETIZING A TRACHEAL REGION OF A PATIENT

(71) Applicant: Edward Vomastek, Traverse City, MI (US)

(72) Inventor: Edward Vomastek, Traverse City, MI (US)

(21) Appl. No.: 13/533,123

(22) Filed: Jun. 26, 2012

Related U.S. Application Data
(60) Provisional application No. 61/502,364, filed on Jun. 29, 2011.

(51) Int. Cl. A61M 16/04

(52) U.S. Cl. CPC A61M 16/0463; A61M 16/0488; A61M 16/0434
USPC 128/200.26

(57) ABSTRACT

A combination stylet and laryngotracheal anesthetizing cannula for insertion within an endotracheal tube during intubation, and which guides the endotracheal tube from its interior during insertion within the patient's trachea, in combination with more efficiently anesthetizing the insertion region both before and during such as the intubation procedure. An attachable vial contains a reservoir of a topical anesthetic or other pharmacological agent, and which attaches to an end of the flexible body in proximity to a stop lock shaped portion, in turn exhibiting an annular end abutment lip or shoulder which, upon insertion within the endotracheal tube, defines an inserting limit to the stylet and outer flexible body. The elongated flexible body defines further includes a narrowed distal extension associated with the topical anesthetizing cannula projecting beyond an end configuration of the main body and, to a limited degree, beyond the distal end of the endotracheal tube.

### CROSS-REFERENCE TO RELATED APPLICATIONS

The present application claims priority of U.S. Provisional Patent Application Ser. No. 61/502,364 filed Jun. 29, 2011.

### FIELD OF THE INVENTION

The present invention relates generally to both form guiding stylet and anesthetizing cannula type devices, such as employed in combination with an endotracheal tube during such as an intubation procedure. More specifically, the present invention discloses a combination anesthetizing cannula and stylet for use with an endotracheal tube during intubation and which guides the endotracheal tube from its interior during insertion within the patient's trachea, in combination with more efficiently anesthetizing the insertion region both before and during such as the intubation procedure.

### BACKGROUND OF THE INVENTION

The prior art is well documented with examples of endotracheal tubes with inner inserted stylets, the purpose for which being to guide the tube during intubation or placement within trachea (windpipe) of the patient in order to maintain an open airway or to serve as a conduit through which to administer certain drugs. An example of a typical endotracheal tube as is known in the prior art is depicted at 1 in FIG. 1 of the drawings and includes an upper proximal end 2 and an inserting and distal end.

Upon being located at the correct position during such as the intubation process, see FIG. 9, An inflatable cuff 4 is expanded by a separate line terminating in a nipple 5 and in order to position the endo tube 1 at the desired location. This is also depicted in the operational view of FIG. 14 and by which the tube 1 is depicted at a desired inserted location of a patient's trachea and retained in place by the admission of pressurized air through the nipple 5, into the connecting line, resulting in the expansion of the inflatable cuff 4.

FIG. 2 presents another prior art illustration of a guiding stylet, at 6, such typically consisting of a malleable metal wire designed to be inserted into the endotracheal tube 1 in order to assist the tube in conforming better to the upper airway anatomy of the specific individual. A generally cylindrical shaped stop lock 7 is provided at the upper distal end of the stylet 6, the purpose of which being to define an abutment stop relative to the inserting upper end 2 of the endotracheal tube 1, thereby prevent over-insertion of the stylet which could result in the stylet projecting beyond the inserting distal end 3 of the tube 1 and risking perforating of the trachea.

FIG. 3 is another prior art illustration of a laryngotracheal cannula device 8, such as including an upper end located plastic or glass cylinder or vial 9 (such as containing a volume of an anesthetic composition not limited to Lidocaine). The cannula 8 further exhibits a plurality of apertures 10 in spaced arrangement proximate a bottom/distal end 11 opposite the vial 9. In use, the cannula is designed to be used prior to placement of the endotracheal tube 1.

### SUMMARY OF THE INVENTION

The present invention combines the attributes of the stylet and laryngotracheal anesthetizing cannula into a combined device for insertion within an endotracheal tube during an intubation procedure, and which guides the endotracheal tube from its interior during insertion within the patient's trachea, this in combination with more efficiently anesthetizing the insertion region both before and during the procedure and as an advantage over such prior art devices which are limited to post insertion anesthesia. The endotracheal stylet extends within a redesigned elongated body which also integrates the topical anesthetizing applying cannula, such that the combination inherits the bendable and form holding properties associated with the stylet.

In one non-limiting application, an attachable vial is provided which contains reservoir of a topical anesthesia such as Lidocaine. The vial attaches to an end of the flexible body in proximity to a stop lock shaped portion in turn exhibiting an annular end abutment lip or shoulder. Upon insertion within the endotracheal tube 1, the elongated flexible body defining the combined device extends within the interior of the outer tube 1, with a narrowed distal extension associated with the topical anesthetizing cannula projecting beyond an end configuration of the main body within which the integrated stylet terminates, the projecting end of the cannula also extending a limited distance beyond the distal inserting end of the endotracheal tube once the device is fed through the interior of the endotracheal tube 1.

In this manner, and by encasing the stylet component within such as a channel or pocket defined in the main flexible body, the necessary form-guiding aspects of the stylet are retained in combination with the ability to anesthetize the tracheal environs during intubation. Additional variants include combining a separate and inner linear extending stylet portion with a second outer form holding stylet material, such as which can be integrated into an outer tubular or sleeve shaped (e.g. cylindrical) body within and through which extend the inner stylet and anesthetizing cannula. In this manner, the combined device can exhibit guiding support to an interior of an endotracheal tube during such as intubation, and while providing progressive topical application of anesthesia such as during and ahead of the progressive installation of the endotracheal tube 1.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a prior art view of an endotracheal tube, similar to what you depict in FIG. 1 of the preceding provisional;

FIG. 2 is a view of an endo-tube form guiding stylet according to the prior art;

FIG. 3 further depicts a prior art laryngotracheal cannula including medicinal charged upper end vessel;

FIG. 4 is a linear cutaway in reduced length of a combination stylet and cannula according to one non-limiting variant of the invention and which includes dual walled stylet portions including an outer stylet portion built into an outer sleeve associated with the supporting body and a separate inner extending style portion terminating at a location proximate a distal endpoint of the outer body;

FIG. 5 is an illustration of the inner extending stylet portion and topical anesthetic delivery cannula, as shown in FIG. 4, and with the outer supporting body with integrated outer stylet portion removed;

FIG. 6 is a linear cutaway in reduced length similar to that shown in FIG. 4 and depicting a further variant in which the inner extending stylet is removed in favor of only the outer extending and integrated stylet also shown in FIG. 4;

FIG. 7 illustrates the inner topical delivery cannula of FIG. 6, with the outer plastic body and integrated stylet being removed;

FIG. 8 is a perspective view of a combination stylet and cannula according to either of the previous embodiments and better depicting the combination of the proximal end mounted medicinal reservoir, the stylet stop lock shaped portion in turn exhibiting an annular end abutment lip or shoulder for engaging the outer endotracheal tube, and the innermost distal and end projecting flexible cannula for delivering topical anesthesia;

FIG. 9 is a combination cannula and stylet according to another possible variant and in which the stylet is configured for telescopic insertion within a reconfigured cannula body;

FIG. 10 is a reduced length linear cutaway of the combination of FIG. 9 depicting the stylet supported in a side-by-side in molded arrangement relative to the cannula for guiding of the outer endo-tube, combined with adequate medicinal delivery along the inserting end of the cannula;

FIG. 11 is a cutaway end view taken along line 11-11 of FIG. 10 and depicting one non-limiting configuration of an inserting distal end of the combination cannula and stylet;

FIG. 12 an illustration similar to FIG. 10 of a combination stylet and cannula according to a further embodiment converging into a generally coaxial type arrangement;

FIG. 13 is an end cutaway view taken along line 13-13 of FIG. 12; and

FIG. 14 depicts an environmental view of an intubation process incorporating the combination cannula and stylet according to any of the previously disclosed embodiments along with the prior art endotracheal tube of FIG. 1.

### DETAILED DESCRIPTION OF THE PREFERRED EMBODIMENTS

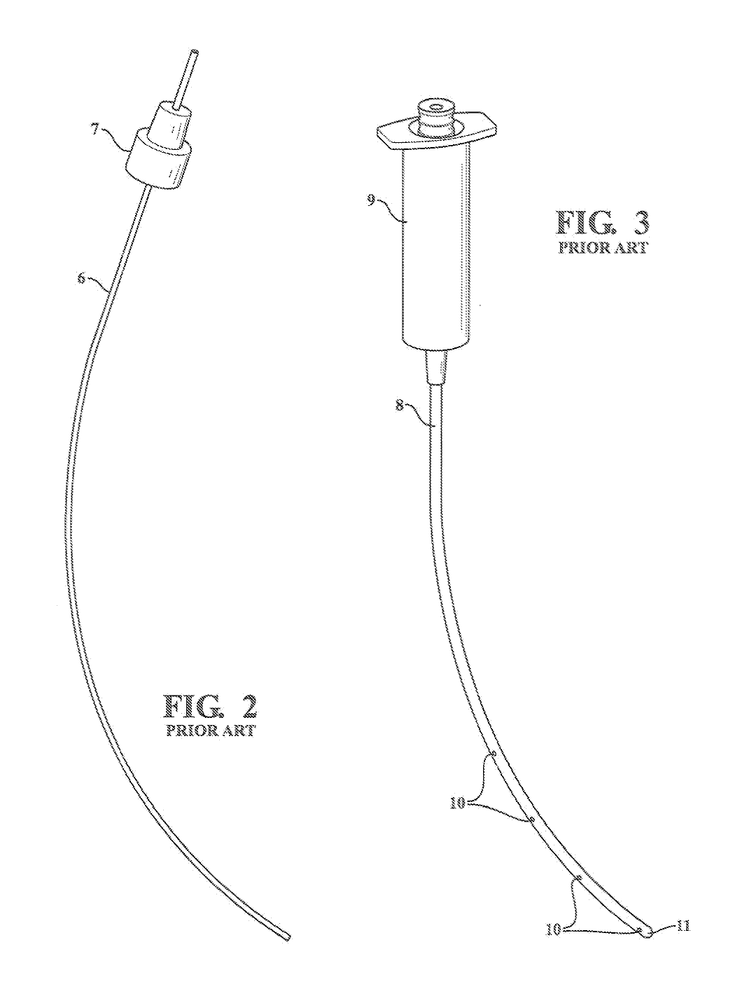

With reference to the various illustrations, the present invention combines the attributes of prior art stylets (see FIG. 2) and laryngo-tracheal anesthetizing cannula (FIG. 3) into a combined device for insertion within an endotracheal tube during intubation. As also described, the combination device manipulates and guides the endotracheal tube from its interior during insertion within the patient's trachea, in combination with more efficiently anesthetizing the insertion region both before and during such as the intubation procedure.

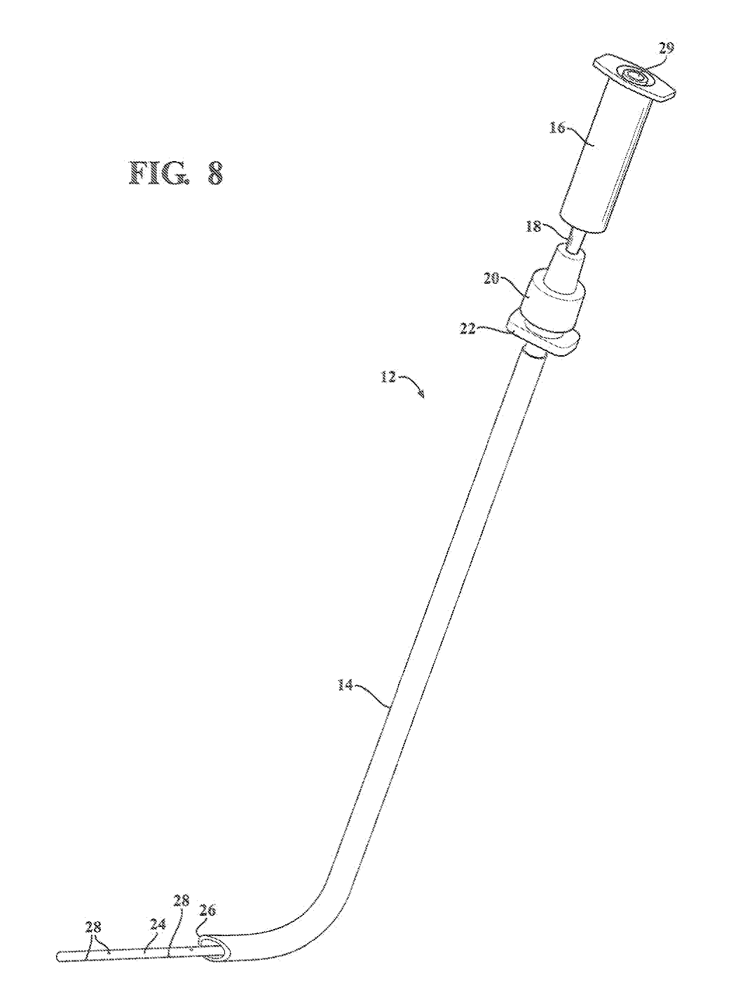

FIG. 8 is a perspective view of a combination stylet and cannula according to any of a number of preferred embodiments. As singularly depicted in the individual stylet 6 and cannula 8 as known in the prior art, the present invention combines these features into a single device, generally shown at 12, in which an outer elongated and sleeve shaped body 14 is provided and which can include a flexible sanitary plastic exhibiting a hollow interior.

A medicinal vial or reservoir is shown at 16 and, similar to the prior art variant 9 depicted in FIG. 3, exhibits a glass or plastic screw in container filled with a volume of such as a fluidic topical anesthetic such as Lidocaine. The present invention also contemplates the use of other pharmacological agents to deliver medication via the trans-tracheal route under certain conditions, and such as where an intravenous route is either unavailable or inappropriate. Without limitation, such an application would be applicable during a “code blue” situation where the patient is in arrest but no intravenous access is present and as an alternative to the trans-endotracheal route utilized in known procedures which is somewhat inefficient for introduction of medications like epinephrine, atropine, etc., this due to absorption into the plastic endotracheal tube.

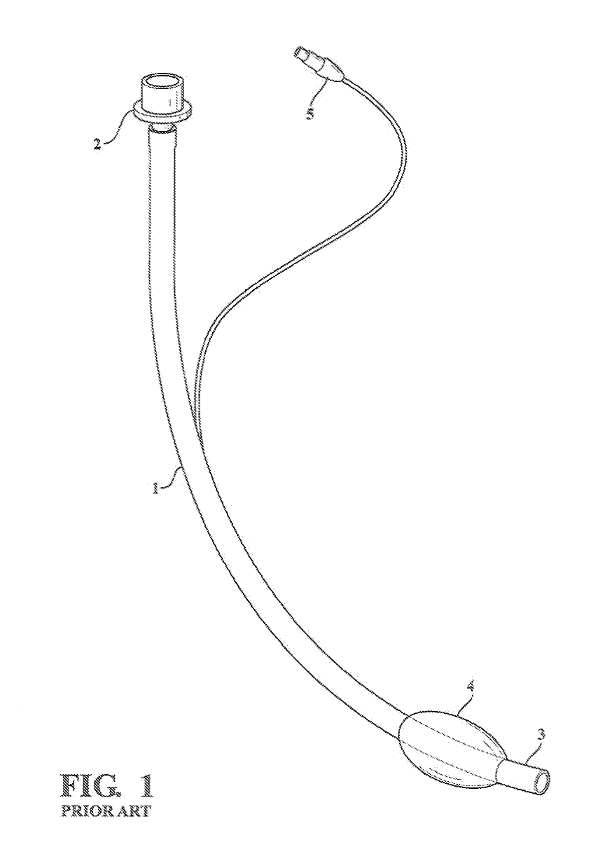

The reservoir 16 can also be affixed by any suitable tab and slot construction or other attachment structure to a proximal (outer) end of the device 12 and, as shown, a narrowed stem or neck 18 inserts into an upper collar location of a proximately located stop lock 20. The stop lock 20 further exhibits an enlarged end face 22 which defines an abutment lip or shoulder when coming into contact with the proximal end 2 of the endotracheal tube 1 again depicted in FIG. 1. An innermost distal end projecting portion 24 is also depicted of a flexible cannula, the portion 24 exhibiting a smaller diameter for projecting beyond an end profile 26 of the outer body 14.

As further shown, the end region of the cannula 24 exhibits a pattern or array of individual apertures 28 defined in any desired arrangement or configuration for facilitating delivery of the topical anesthesia communicated from the upper end located vial 16 (and such as further upon engaging a plunger or like component through an accessible end location 29 for influencing the anesthesia to travel linearly through the interior of the body 12 and out the apertures 28 in the distal projecting end of the cannula 24.

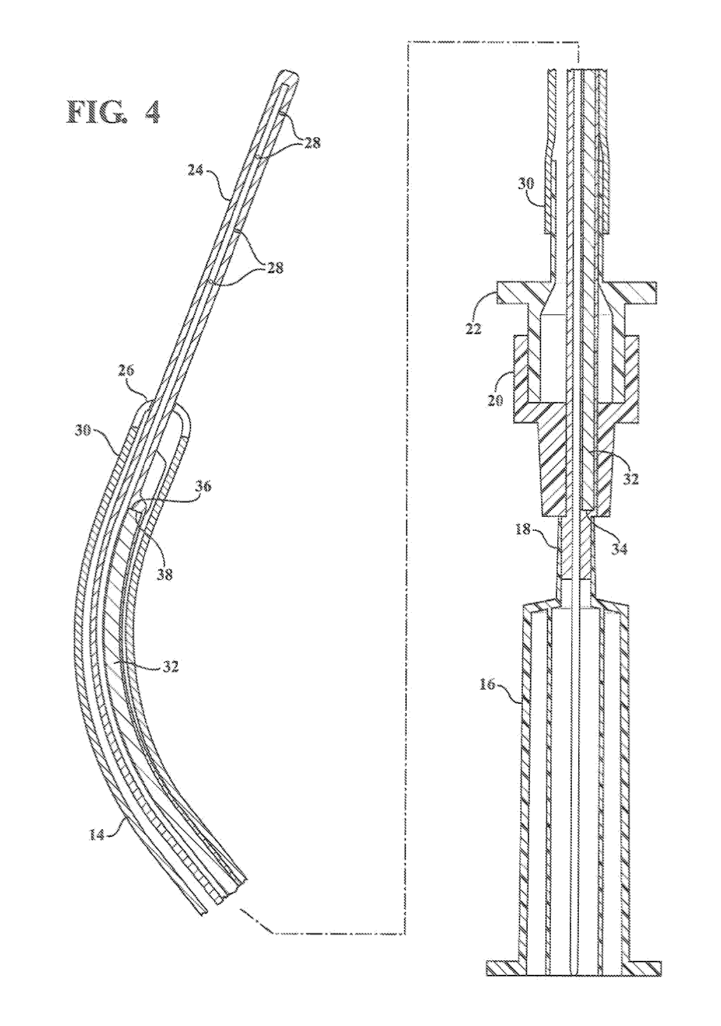

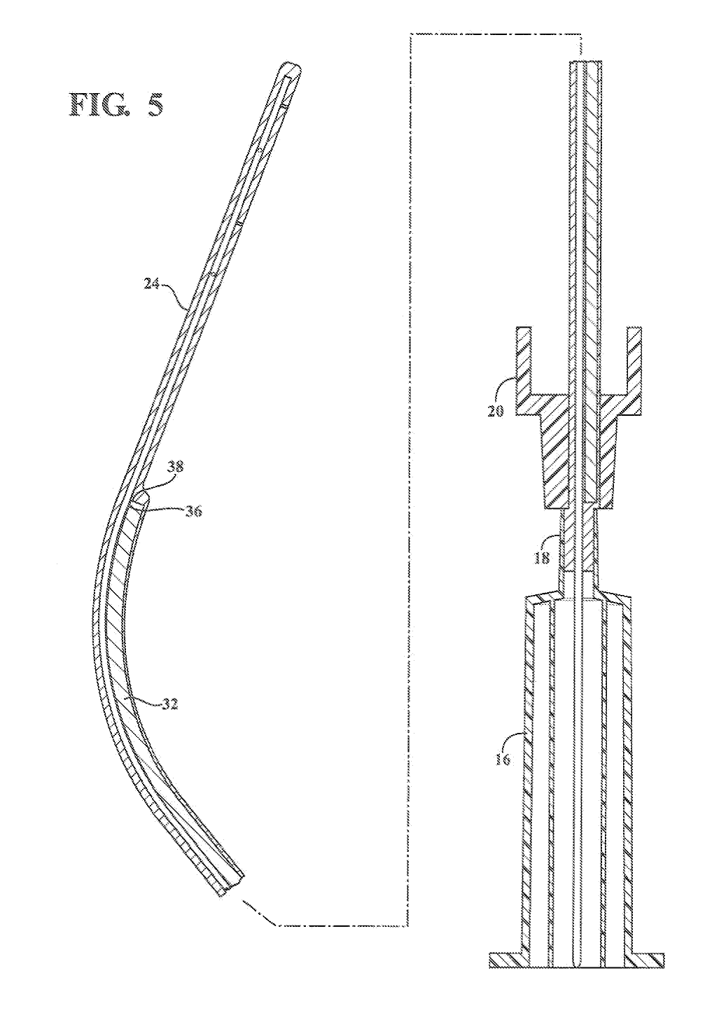

Referring now to FIGS. 4-7, a pair of related variants are illustrated in cutaway of potential constructions of the elongated device 12, it being understood that the present invention contemplates any number of constructions in which the features of a form-guiding stylet along with that of a medicinal delivery cannula are combined into a common device for providing guiding of the endotracheal tube 1 during intubation along with both pre and post anesthetizing of the patient's trachea. As will be further explained, the present invention contemplates any of a variety of different configurations for combining the features of a form-holding and supporting stylet with those of the plastic flexible body and which are not limited to those particular variants depicted.

Initially referencing FIG. 4, a linear cutaway is depicted in reduced length of a combination stylet and cannula according to one non-limiting variant of the invention. The device depicts an arrangement of dual walled stylet portions in which an outer stylet portion 30 is built into a cylindrical extending wall associated with the outer supporting body 14, along with a separate inner extending style portion 32 extending in coaxially interior extending fashion from a proximal end location 34 proximate the stop lock 20 to a distal end location 36 proximate the distal endpoint 26 of the outer body.

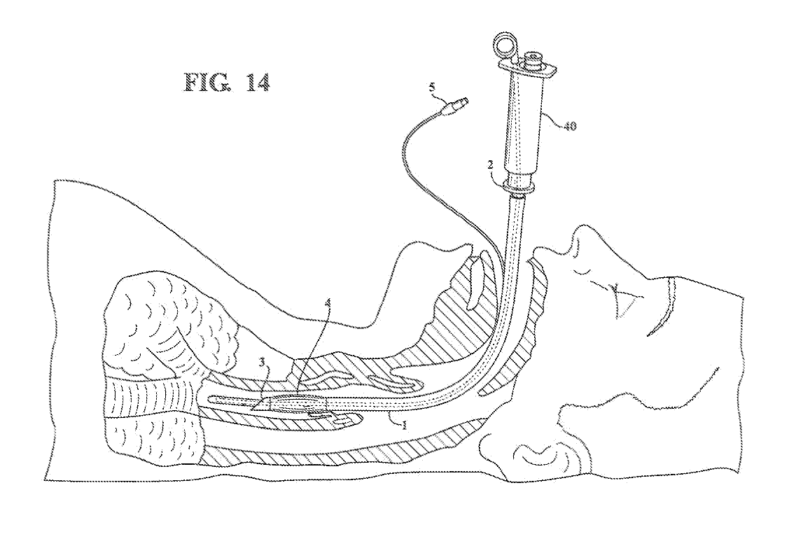

By way of further explanation, the outer stylet configuration 30 is understood to include any type of mesh grid, coil or weave construction which can be integrated into the sleeve shaped wall of the outer silastic (i.e. defined as sanitary and flexible plastic) body 14 of the device, such as which can occur during a suitable molding or extrusion process during which the body is formed as part of a manufacturing process for creating the overall device. It is further understood that the present invention contemplates any arrangement of form holding and guiding stylets, these typically being constructed of a malleable and bendable (soft) metal which can be manipulated in order to provide a desired degree of interior support to the endotracheal tube during manipulation in the intubation process (reference also being had to the operational view of FIG. 14).

As again shown in FIG. 4 and successive FIG. 5, the inner stylet 32 exhibits an elongated bar or rod shape with its distal end 36 abutting against a shoulder 38 associated with the inner cannula 24. FIG. 5 is again an illustration of the inner extending stylet portion 32 and topical anesthetic delivery cannula 24, as also shown in linear cutaway in FIG. 4, and with the outer supporting body 14 with integrated outer stylet portion 30 removed along with the endotracheal tube contacting end face 22 removed.

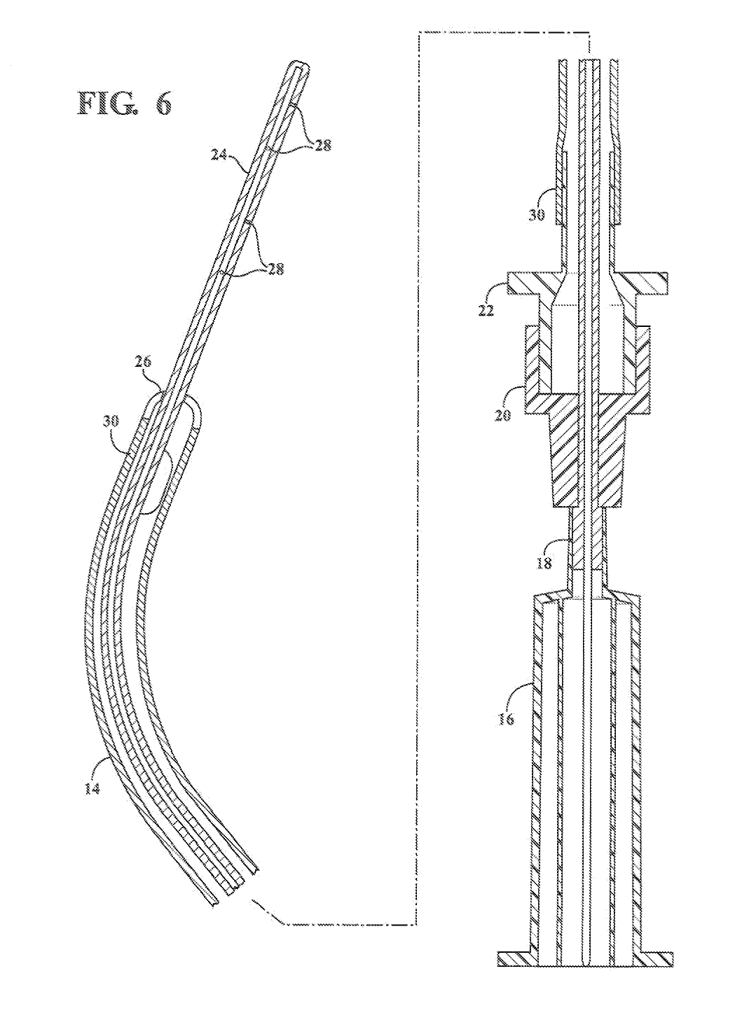

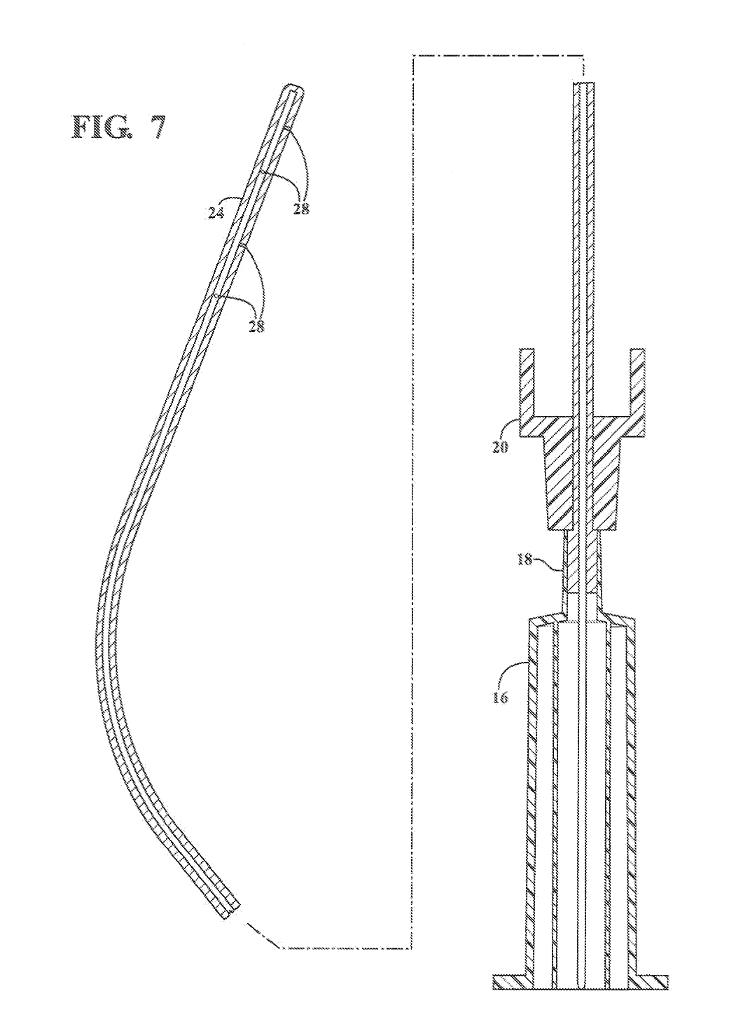

FIG. 6 is a linear cutaway in reduced length similar to that shown in FIG. 4 and depicting a further variant of the combination device and in which the inner extending stylet is removed in favor of only the outer extending and integrated stylet 30. FIG. 7 further illustrates the inner topical delivery cannula 24 of FIG. 6, with the outer plastic body 14 and integrated stylet removed.

It is again worth mentioning that the design of the stylet stop lock (again including pseudo cylindrical projection 20 and enlarged end face 22) can be redesigned or modified from that shown and in any manner desired in order to provide desired contact to the opposing proximal end face (again at 2 in FIG. 1) of the flexible endotracheal tube 1. The linear cutaway illustrations of FIGS. 4-7 of the related variants also depict the manner in which the communicating interior passageway within the elongated body communicates the proximal end attachable medicinal vial 16 with the distal end located apertures 28 arranged in the cannula 24 in order to provide a desired topical application of the anesthetic.

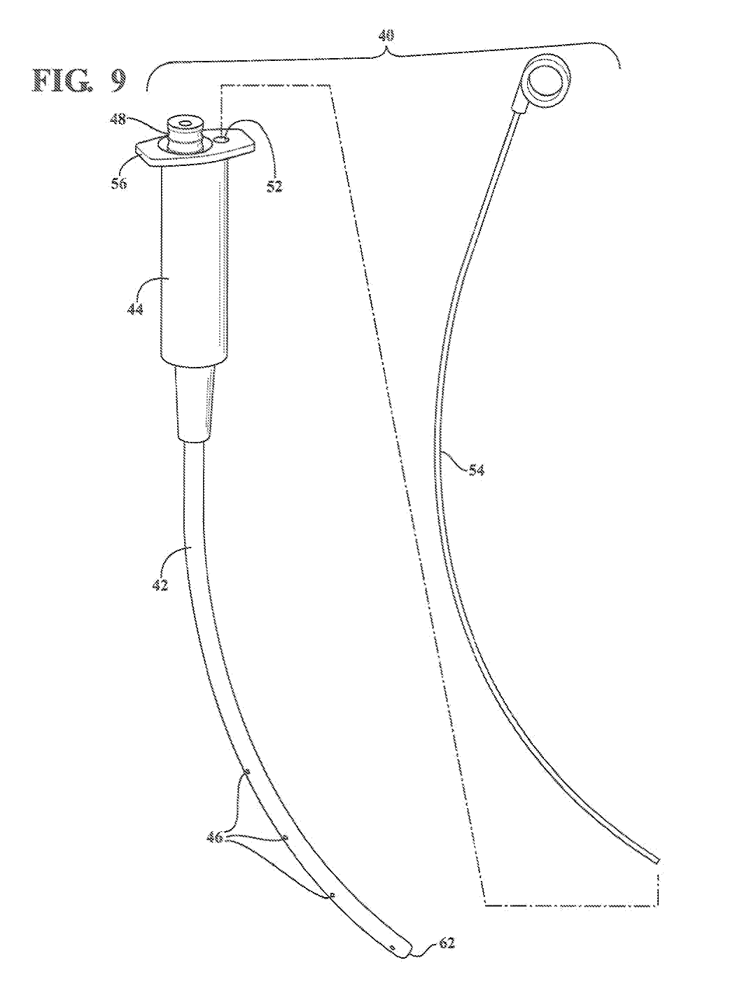

Proceeding on to FIG. 9, a combination cannula and stylet is generally depicted at 40 according to another possible variant in comparison to the variants depicted and previously described in FIGS. 4-8. A redesigned cannula includes an elongated and flexible body 42, to which at a proximal end is secured a medicinal vial 44.

The cannula 42 includes an upper pathway section 45 which extends within the vial 44 and communicates with a further pathway 50 terminating, at a distal proximate end, in a pattern or array of apertures 46 such as which can be configured in either or both linear and circumferentially offset fashion proximate the distal end of the cannula in order to communicate the anesthetic in any desired mist, spray or finely apportioned and atomized fashion to the topical region of the trachea during intubation. Also shown at 48 is an upper end nipple location for assisting in injecting medicine within and through the vial 44, into the cannula 42, and out through the distal end located apertures 46.

Astride a first communicating pathway 50 designed into the cannula 42 for feeding the apertures 46 is a second inner channel or pathway 52 for receiving in telescopically inserting fashion a stylet 54 (again FIG. 9). The pathway 52 extends from an upper proximal location of the cannula exhibited by an enlarged lip edge 56 (such as for engaging in abutting fashion the proximal end of the endotracheal tube 1) to a distal end location 58 which is astride a distal end 60 of the main communicating pathway.

As further depicted in cutaway in FIG. 10, a bottom most distal location 62 of the device is closed in order to prevent the inserted stylet 54 from coming into contact with the patient trachea. It is further envisioned and understood that the stylet 54 and associated pathway 52 can be redesigned such that it terminates at a position short of the distal end 62 similar to that depicted in the variants shown in FIGS. 4-8.

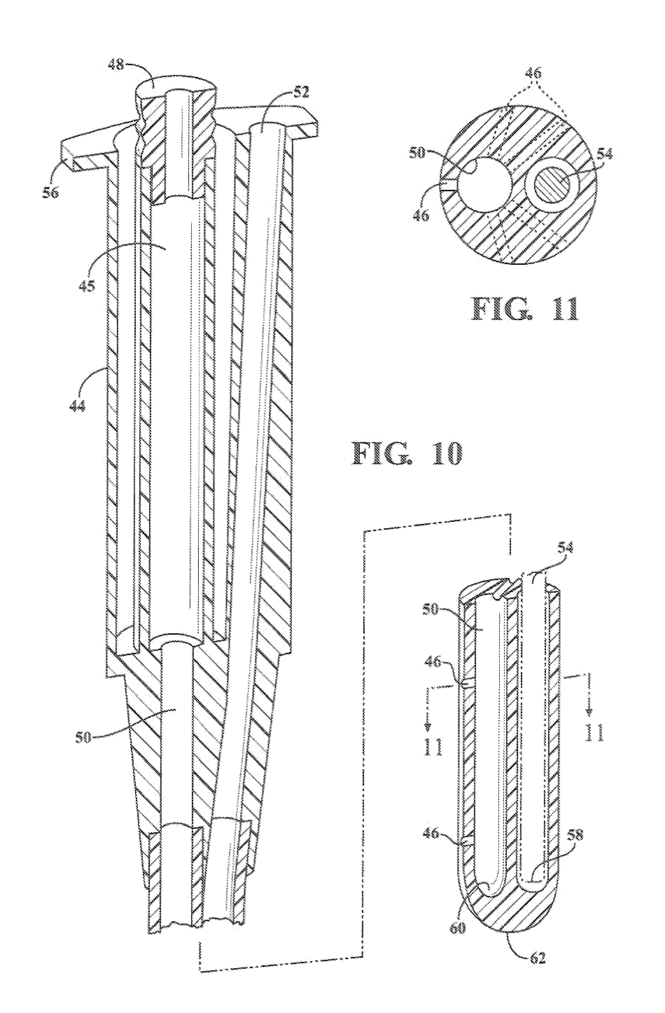

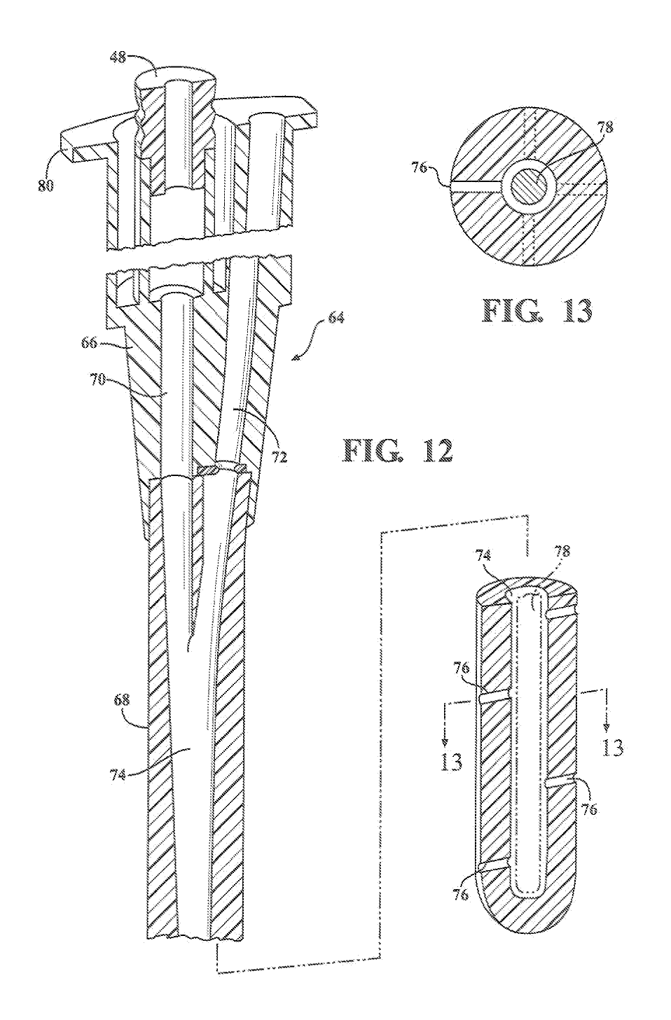

FIG. 12 is an illustration similar to FIG. 10 of a combination stylet and cannula, generally at 64, and according to a further embodiment. The device 64 includes an integrated vial 66 attached to an upper end of a modified cannula 68 which differs from that previously shown in cutaway in FIGS. 10-11 in that individual pathways 70 (medicinal delivery) and 72 (style) converge into a single pathway 74 in communication with an array of distal end located a delivery apertures 76.

A stylet 78 (see in phantom representation 8 in FIG. 12 and in solid cross sectional depiction in FIG. 13) is inserted into the upper passageway 72 and communicates into the conjoined lower passageway 74 in such a fashion that it retains a desired spacing between the outer surface of the stylet 78 and the inner closed surfaces of the passageway 74 in order to both enable a reduction in diameter of the in-fed cannula 68 as well as provide better manipulation of the device when inserted inside of the endotracheal tube 1. Other features also shown in the variant of FIG. 10 include an enlarged lip edge 80 for seating against the proximal end of the endotracheal tube 1, as well as again the provision of the upper end nipple location 48 for assisting in injecting medicine within and through the vial 66, into the cannula 68, and out through the distal end located apertures 76.

Finally, FIG. 14 depicts an environmental view of an intubation process incorporating the combination cannula and stylet according to any of the previously disclosed embodiments (and without limitation depicting by example the variant of the device shown at 40 in FIG. 9) along with the prior art endotracheal tube 1 of FIG. 1.

Having described my invention, other and additional preferred embodiments will become apparent to those skilled in the art to which it pertains, and without deviating from the scope of the appended claims. This can, at a minimum, include variations of the form guiding stylet as best depicted in the initially depicted embodiments of FIGS. 4-8 being redesigned as a single coiled configuration either built into the sleeve shape of the cannula or constructed so as to extend within a linear extending interior passageway of the cannula in such a fashion as not to impede the delivery of medication from the associated vial through the cannula body and out through the distal end arrayed aperture pattern.

1. A combination laryngotracheal cannula and stylet device for use in guiding an outer endotracheal tube during an intubation procedure, comprising:
an elongated body having a communicating interior extending between a topical anesthesia holding reservoir at a proximal end and a delivery mechanism at a distal end; and
a bendable and form holding stylet extending within said body from said proximal end to a location short of said distal end;
said body adapted to being inserted within an interior of the endotracheal tube during intubation such that said delivery mechanism projects a distance beyond the tube to administer the topical anesthesia prior to and during location of the tube.

2. The device as described in claim 1, further comprising a modified cylindrical shaped stop lock located at said proximal end of said body and which is adapted to contact a proximal end location of the outer tube in order to limit an inserting distance of said body within the tube.

3. The device as described in claim 1, said reservoir further comprising an attachable glass or plastic body.

4. The device as described in claim 1, said distal end of said body further comprising a flexible tip, said delivery mechanism including a plurality of ports extending through said tip for delivering the topical anesthesia.

5. The device as described in claim 1, said stylet further comprising an outer portion integrated into a cylindrical extending wall associated with said body.

6. The device as described in claim 2, said stylet further comprising an inner portion extending in coaxially interior extending fashion from a proximal end location of said stop lock to a distal end location proximate a distal endpoint of said body.

7. The device as described in claim 1, further comprising said delivery mechanism being integrated into a narrowed diameter flexible cannula projecting beyond a distal end of an outer elongated portion of said body.

8. The device as described in claim 1, further comprising a first medicinal delivery pathway and a second spaced apart and stylet receiving pathway extending between said proximal and distal ends of said body.

9. The device as described in claim 1, further comprising a first medicinal delivery pathway and a second spaced apart and stylet receiving pathway extending from said proximal end and conjoining into a common pathway to said distal end of said body.

10. A combination laryngotracheal cannula and stylet device for use in guiding an outer endotracheal tube during an intubation procedure, comprising:
an elongated body having a cylindrical extending wall defining a communicating interior and extending between a topical anesthesia holding reservoir at a proximal end and a delivery mechanism at a distal end;
a bendable and form holding stylet integrated into said wall associated with said body terminating short of said delivery mechanism; and
said delivery mechanism including a plurality of ports delivering the topical anesthesia such that, upon said body adapted to being inserted within an interior of the endotracheal tube during intubation, said delivery mechanism projecting a distance beyond the tube to administer the topical anesthesia prior to and during intubating location of the tube.

11. The device as described in claim 10, said stylet further comprising a second elongated and inner extending portion arranged in coaxially interior extending fashion relative to wall integrated stylet, said inner style extending from a proximal end location of said body to a distal end location proximate a distal endpoint of said body.

12. The device as described in claim 10, further comprising a modified cylindrical shaped stop lock located at said proximal end of said body and which is adapted to contact a proximal end location of the outer tube in order to limit an inserting distance of said body within the tube.

13. The device as described in claim 10, said reservoir further comprising an attachable glass or plastic body.

14. The device as described in claim 10, said distal end of said body further comprising a flexible tip, said delivery mechanism including a plurality of ports extending through said tip for delivering the topical anesthesia.

15. The device as described in claim 10, further comprising said delivery mechanism being integrated into a narrowed diameter flexible cannula projecting beyond a distal end of an outer elongated portion of said body.
